(19) United States
(12) Patent Application Publication
(10) Pub. No.: US 2014/0155966 A1
(43) Pub. Date: Jun. 5, 2014

(54) IMPLANTABLE LEAD WITH BODY PROFILE OPTIMIZED FOR IMPLANT ENVIRONMENT

(71) Applicant: PACESETTER, INC., Sylmar, CA (US)

(72) Inventors: Dorab N. Sethna, Culver City, CA (US); Steven R. Conger, Agua Dulce, CA (US)

(73) Assignee: PACESETTER, INC., Sylmar, CA (US)

(21) Appl. No.: 13/691,028

(22) Filed: Nov. 30, 2012

(51) Int. Cl. A61N 1/05

(52) U.S. Cl. CPC A61N 1/05
USPC 607/116; 156/60

(57) ABSTRACT

Implementations described and claimed herein provide an implantable lead optimized for an implant environment and methods of manufacturing such implantable leads. The implantable lead includes an insulation layer having one or more transitions along a length of the insulation layer from a proximal end to a distal end. Each of the transitions is a seamless change from a section of the insulation layer having a set of performance characteristics to another section of the insulation layer having a different set of performance characteristics.

### FIELD OF THE INVENTION

Aspects of the presently disclosed technology relate to medical apparatuses and methods. More specifically, the presently disclosed technology relates to implantable medical leads and methods of manufacturing such leads.

### BACKGROUND OF THE INVENTION

Implantable medical devices are widely used for electrically stimulating body tissue and/or sensing the electrical activity of such tissue. Such devices include, without limitation, pacemakers, defibrillators, cardioverters, neurostimulators, etc. Generally, implantable medical devices include a pulse generator electrically coupled to one or more leads carrying electrode(s). Various lead types for different placement approaches have been developed. However, many of these lead types are susceptible to reliability issues and/or inferior biostability depending on the environment in which the lead is implanted.

Lead insulation abrasion and crush failures are common reliability issues. Specifically, frictional contact and harsh implant environments can abrade lead insulation or crush a lead, resulting in lead failure, which could expose conductors and/or cause the implantable medical device to: experience a short; improperly sense the electrical activity of body tissue; deliver an inappropriate therapy; fail to deliver a therapy when needed; or experience other failures. Some leads include an insulation layer made from a durable material, such as polyurethanes (e.g., Pellethane 80A or 55D), to reduce the propensity of abrasion and crush failures. However, such polyurethane insulation layers often increase lead body stiffness, which may increase the risk of trauma to implant environments more susceptible to perforations, and have significantly reduced biostability. For example, the right ventricular apex of the heart is relatively thin, so using a lead having a relatively stiff body increases the risk of puncturing the right ventricular apex. On the other hand, leads including an insulation layer made from a flexible material, such as silicone, that renders the leady body generally a-traumatic to implant environments more susceptible to perforations often perform poorly under abrasion and crush forces.

Some leads have been developed that include a co-polymer insulation layer that compromises between these features of polyurethane insulation layers and silicone insulation layers. However, insulation layers are conventionally applied in as-extruded tube form from end to end. Stated differently, insulation layers are limited to a uniform body profile (e.g. a thin-walled body profile or a thick-walled body profile) from a proximal end of the lead to a distal end of the lead. As such, although the proximal and distal ends of a lead generally demand conflicting mechanical properties based on implant environment, such insulation layers are limited to uniform properties from end to end that are a compromise between the properties suitable for the proximal end and the properties suitable for the distal end. Specifically, the distal end of most leads is sensitive to stiffness, particularly when used in implant environments susceptible to perforations, so maximized flexibility of the lead body is desirable at the distal end. Conversely, the proximal end of most leads is sensitive to abrasion and crush forces, while being less sensitive to stiffness, and therefore, maximized durability and resilience is desirable at the proximal end. While a uniform thin-walled body profile ensures that lead body stiffness remains within acceptable limits and thus is suitable for the distal end, a uniform thin-walled body profile has reduced resilience to abrasion and crush forces. On the other hand, a uniform thick-walled body profile is more resilient to abrasion and crush forces, which is suitable for the proximal end, but at the cost of flexibility at the distal end.

Accordingly, there is a need in the art for an implantable lead that provides lead body flexibility while increasing resilience to reliability concerns, such as abrasion, crush, or other insulation failures, depending on the environment in which a section of the implantable lead is to be implanted. There is also a need in the art for a method of manufacturing such an implantable lead.

### BRIEF SUMMARY OF THE INVENTION

Implementations described and claimed herein address the foregoing problems by providing an implantable lead with a body profile having a plurality sections each optimized for an environment in which the section is to be implanted. In one implementation, the implantable lead includes an insulation layer having one or more transitions along a length of the insulation layer from a proximal end to a distal end. Each of the transitions is a seamless change from a section of the insulation layer having a set of performance characteristics to another section of the insulation layer having a different set of performance characteristics.

A method for manufacturing such implantable leads is also disclosed herein. In one implementation, a plurality of insulation layer sections are obtained. Each of the insulation layer sections has a set of performance characteristics based on a local environment in which the insulation layer section is to be implanted. The plurality of insulation layer sections are positioned relative to each other and fused together such that one or more transitions are formed along a length of a composite insulation layer.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 is a diagrammatic depiction of an electrotherapy system electrically coupled to a patient heart, as shown in an anterior view, a distal portion of each lead being implanted in the patient heart.

FIG. 2A is a longitudinal cross-sectional elevation of an implementation of an implantable lead with a body profile having a plurality of sections, each section optimized for an environment in which the section is to be implanted.

FIG. 2B is a transverse cross-sectional elevation of the lead of FIG. 2A as taken along section line 2B-2B of FIG. 2A.

FIG. 3A shows a longitudinal elevation of a first insulation layer section having a first set of performance characteristics and a second insulation layer section having a second set of performance characteristics placed over a support mandrel.

FIG. 3B is the same view as FIG. 3A with the first and second insulation layers fused together to form an insulation layer with a seamless transition between the first and second sections.

FIG. 3C shows the same view and FIG. 3B with the support mandrel removed.

FIG. 4 illustrates example operations for manufacturing an implantable lead with a body profile having one or more sections, each section optimized for an environment in which the section is to be implanted.

FIG. 5 illustrates an implementation of an implantable lead having an insulation layer with one or more sections optimized for implant in the right atrium.

FIG. 6 shows an implementation of an implantable lead having an insulation layer with one or more sections optimized for implant in the right ventricle.

FIG. 7 shows an implementation of a defibrillation lead having an insulation layer with one or more sections optimized for implant in the right ventricle.

FIG. 8 illustrates an implementation of a transvenous CRT lead having an insulation layer with one or more sections optimized for implant in the left ventricle.

FIG. 9 is a lead generally the same as the lead of FIG. 8, except the lead body transitions to enlarged diameters at the location of one or more of the ring electrodes supported on the lead body.

FIG. 10 shows illustrates an implementation of an implantable lead having an insulation layer with one or more sections optimized for implant on the epicardium.

### DETAILED DESCRIPTION

Aspects of the presently disclosed technology involve implantable medical leads with a body profile having a plurality sections each optimized for an environment in which the section is to be implanted and methods of manufacturing such implantable medical leads. In one aspect, the implantable medical lead includes an insulation layer having one or more seamless transitions in performance characteristics (e.g., thickness, material type, etc.) along a length of the insulation layer between a proximal end and a distal end. The transitions create a plurality of sections, each section optimized for the environment in which the section will be implanted without compromising the performance of an adjacent section. For example, the insulation layer may have a transition between a thin-walled insulation section at the distal end, where lead-body flexibility is desirable, and a thick-walled insulation section at the proximal end, where abrasion, crush, and wrinkle/crack resistance is needed. Various example implementations of the implantable medical lead optimized for a variety of implant environments and placement approaches are disclosed herein.

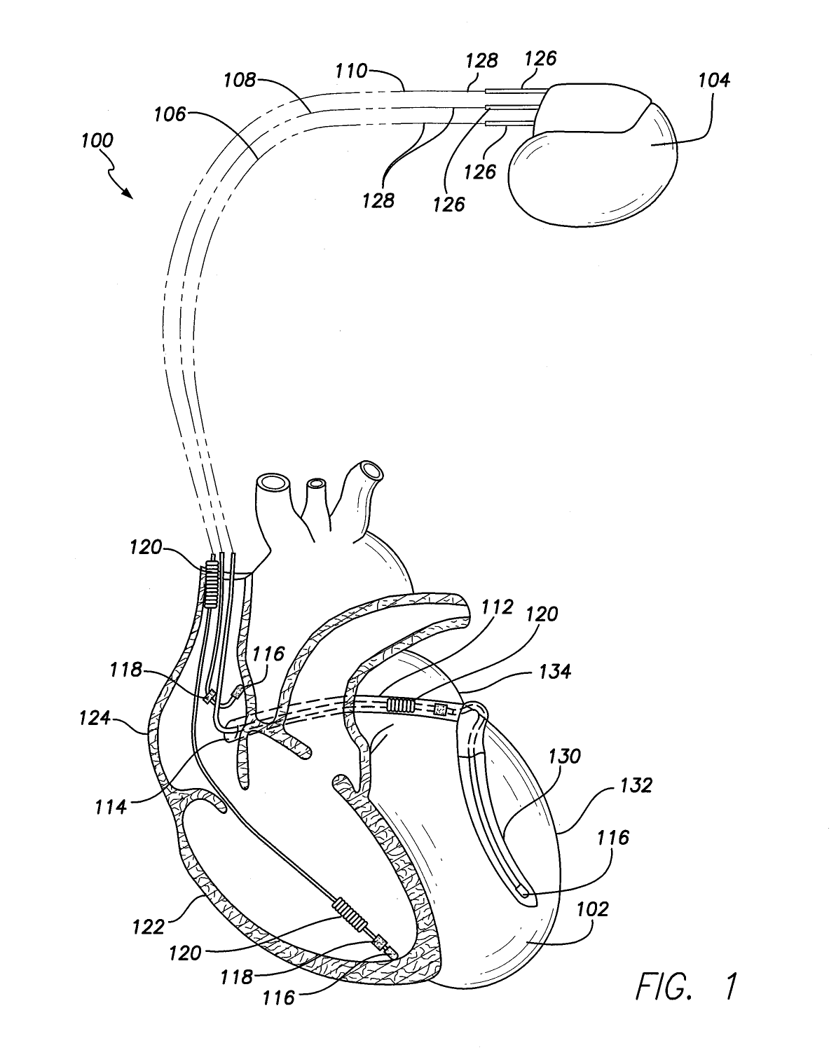

To begin a general, non-limiting discussion regarding some of the features and deployment characteristics common among the various implantable lead implementations disclosed herein, reference is made to FIG. 1, which is a diagrammatic depiction of an electrotherapy system 100 electrically coupled to a patient heart 102. As shown in FIG. 1, the electrotherapy system 100 includes an implantable pulse generator 104, which may be, for example, a pacemaker, an implantable cardioverter defibrillator (“ICD”), or other device for electrically stimulating body tissue and/or sensing the electrical activity of such tissue. The electrotherapy system 100 includes one or more implantable medical leads (e.g., a left ventricular (“LV”) lead 106, a right ventricular (“RV”) lead 108, and a right atrial (“RA”) lead 110) electrically coupling the patient heart 102 to the pulse generator 104. The implantable medical leads 106, 108, and 110 each have a body profile comprising a plurality sections each optimized for an environment in which the section is to be implanted. In the example shown in FIG. 1, the implantable medical leads 106, 108, and 110 include a plurality of sections optimized for implant in the left ventricle 132, right ventricle 122, and right atrium 124, respectively. As described herein, the implantable medical leads 106, 108, and 110 each include an insulation layer having one or more seamless transitions in performance characteristics along a length of the insulation layer between a proximal end 128 and a distal end 116.

As can be understood from FIG. 1, which shows an anterior view of the patient heart 102, the coronary sinus 112 extends generally patient right to patient left from the coronary sinus ostium 114 and posterior to anterior until transitioning into the great cardiac vein 130, which then extends in a generally inferior direction along the anterior region of the left ventricle 132. In extending generally posterior to anterior from the coronary sinus ostium 114 until transitioning into the great cardiac vein 130, the coronary sinus 112 is inferior to the left atrium 134 and superior to the left ventricle 132. When implanted, the LV lead 106 extends through the coronary sinus 112 via the coronary sinus ostium 114 into the great cardiac vein 130 to provide electrical stimulation of the basal region of the patient heart 102. The RV and RA leads 108 and 110 are placed to provide electrical stimulation to the right ventricle 122 and the right atrium 124, respectively.

The implantable medical leads 106, 108, and 110 may employ pacing electrodes, as shown in FIG. 1 at the distal ends 116, sensing electrodes 118, and shock coils 120 to provide electrical stimulation to the patient heart 102. Further, each of the leads 106, 108, and 110 is electrically coupled to the pulse generator 104 via a lead connector end 126 at the lead proximal end 128. Electrical conductors extend through each lead body from electrical contacts on the lead connector end 126 to the various electrodes 116 and 118 and the shock coils 120 to provide electrical communication with the pulse generator 104. The electrical conductors are covered by an insulation layer having one or more seamless transitions in performance characteristics along a length of the insulation layer to provide a lead body having one or more sections optimized for the implant environment.

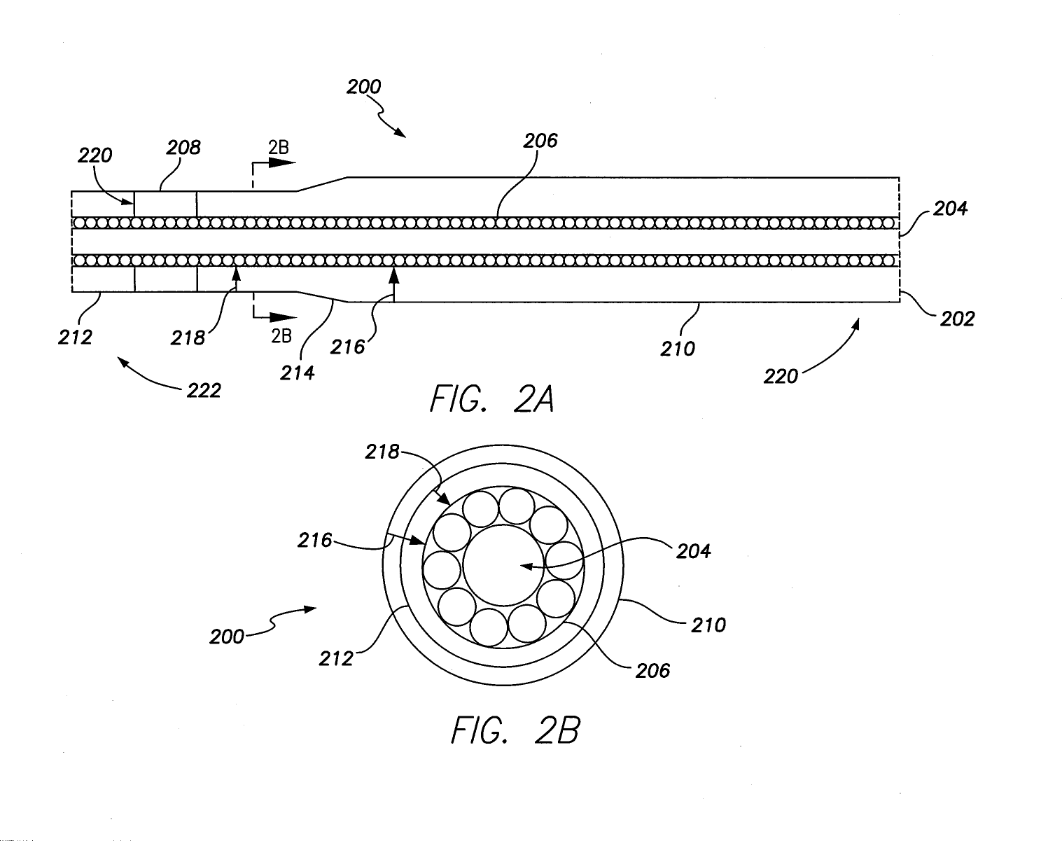

Turning to FIGS. 2A and 2B, a detailed description is provided of an implementation of an implantable lead 200 with a body profile having a plurality of sections, each section optimized for a local environment in which the section is to be implanted. In one implementation, the implantable lead 200 includes an insulation layer 202 encapsulating a central lumen 204 and electrical conductors 206.

As can be understood from FIGS. 2A and 2B, while the insulation layer 202 is continuous, a profile of the insulation layer 202 varies along the length of the insulation layer 202 from a proximal end 220 to a distal end 222. Specifically, the insulation layer 202 includes a transition 214 between a first section 210 having a first set of performance characteristics and a second section 212 having a second set of performance characteristics that is different from the first set of performance characteristics. The transition 214 from the first section 210 to the second section 212 is seamless. Further, the transition 214 ensures that the first set of performance characteristics are optimized for a first local environment in which the first section 210 is to be implanted without compromising the second set of performance characteristics, which are optimized for a second local environment in which the second section 212 is to be implanted. Accordingly, the insulation layer 202 is optimized to meet the demands of varying local implant environments traversed in an implant path and to provide positive contact of various components of the implantable lead 200 with surrounding body tissue in a given local implant environment.

In one implementation, the first and second sets of performance characteristics include wall thickness, material type, and/or durometer. As shown in the implementation illustrated in FIGS. 2A and 2B, the first section 210 has a first wall thickness 216 and the second section 212 has a second wall thickness 218 that is different than the first wall thickness 216. For example, the first wall thickness 216 may be larger to increase abrasion, crush, and wrinkle/crack resistance, and the second wall thickness 218 may be smaller to increase lead-body flexibility. Further, in some implementations, the first section 210 is made from a different material having a different durometer than that of the second section 212. For example, the first section 210 may be made from a thermoplastic material having a higher durometer, such as Pellethane 55D, to increase abrasion, crush, and wrinkle/crack resistance, and the second section 212 may be made from a thermoplastic material having a lower durometer, such as Elasteon-2A, to increase lead-body flexibility. Other materials include, but are not limited to, polyurethane, silicone, polystyrene-isobutylene-styrene (PIBS), fumed silica, Optim™, soft-Optim™, CarboSil®, Tecothane®, and other polymers.

Although the implementation shown in FIGS. 2A and 2B includes one transition, it will be understood by those skilled in the art that the insulation layer 202 may comprise additional transitions along the length of the insulation layer 202 from the proximal end 220 to the distal end 222 between additional sections, each having performance characteristics optimized for the environment in which the section is to be implanted. For example, where an implant environment warrants increased robustness, stiffness, and/or abrasion, crush, or wrinkle/crack resistance, such as in the pocket area, tunneled path, around bones (e.g., clavicle or ribs), or in vasculature (e.g., cephalic vein, sub-clavian vein, or superior vena cava), a section to be implanted in that environment has a set of performance characteristics optimized for that implant environment. Similarly, where an implant environment warrants increased flexibility, a section to be implanted in that environment has a set of performance characteristics optimized accordingly.

The insulation layer 202 encapsulates and protects the central lumen 204 and the electrical conductors 206. The central lumen 204 may be used to insert or inject, for example, a guide wire, a structure with a deployable electrode or sensor, a contrast fluid to facilitate fluoroscopic viewing, a fixation mechanism, and/or an extraction mechanism. The electrical conductors 206 electrically couple one or more electrodes (e.g., electrode 208) to a pulse generator to electrically stimulate body tissue and/or sense the electrical activity of such tissue. The electrical conductors 206 may include, without limitation, wires, cables, or helically coiled filars. In the example shown in FIG. 2A, the electrode 208 is created by placing an annular ring 208 formed of an electrically conductive material (e.g., platinum, platinum-iridium alloy, stainless steel, etc.) in a void region 220 of the insulation, the void 220 being created by removing an annular portion of the insulation layer 202 to expose a portion of the electrical conductors 206. The ring 208 is electrically coupled to the exposed portion of the electrical conductors 206. However, it will be appreciated by those of ordinary skill that the position and type of the electrode 208 may vary.

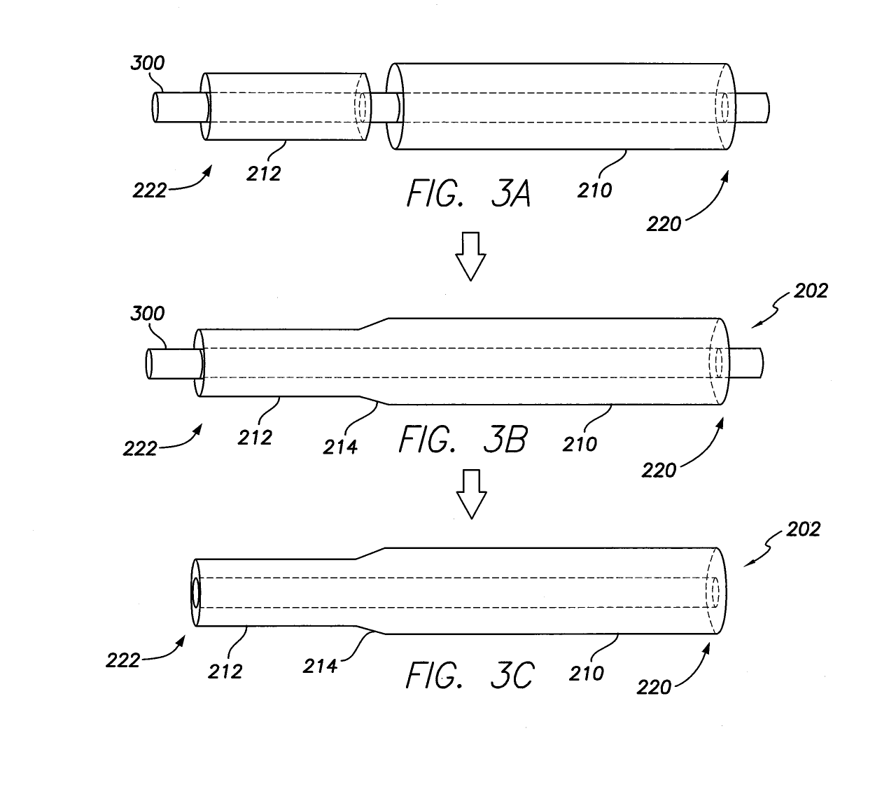

To begin a detailed discussion of methods for manufacturing the implantable lead 200, reference is made to FIGS. 3A-3C. As can be understood from FIG. 3A, the first section 210 and second section 212 are placed over a support mandrel 300. As discussed above, the first set of performance characteristics of the first section 210 are different from the second set of performance characteristics of the second section 212. The sections 210 and 212 are each placed on the support mandrel relative to each other based on a profile of the insulation layer 202 needed to meet the demands of various local implant environments. Stated differently, the sections 210 and 212 are placed on the support mandrel 300 relative to each other such that the insulation layer 202 that is formed positions the sections 210 and 212 along the length of the insulation layer 202 to correspond to the local environment in which the sections 210 and 212 will be implanted.

For example, as shown in FIG. 3A, where the second section 212 has the second set of performance characteristics optimized for lead-body flexibility, the second section 212 is placed on the support mandrel 300 at what will become the distal end 222 of the insulation layer 202, and where the first section 210 has the first set of performance characteristics optimized for robustness, stiffness, and/or resistance to reliability issues, the first section 212 is placed on the support mandrel 300 at what will become the proximal end 220. Additional sections having the same or different performance characteristics as either the first section 210 or the second section 212 may also be placed over the support mandrel 300 based on the local environment in which the sections will be implanted.

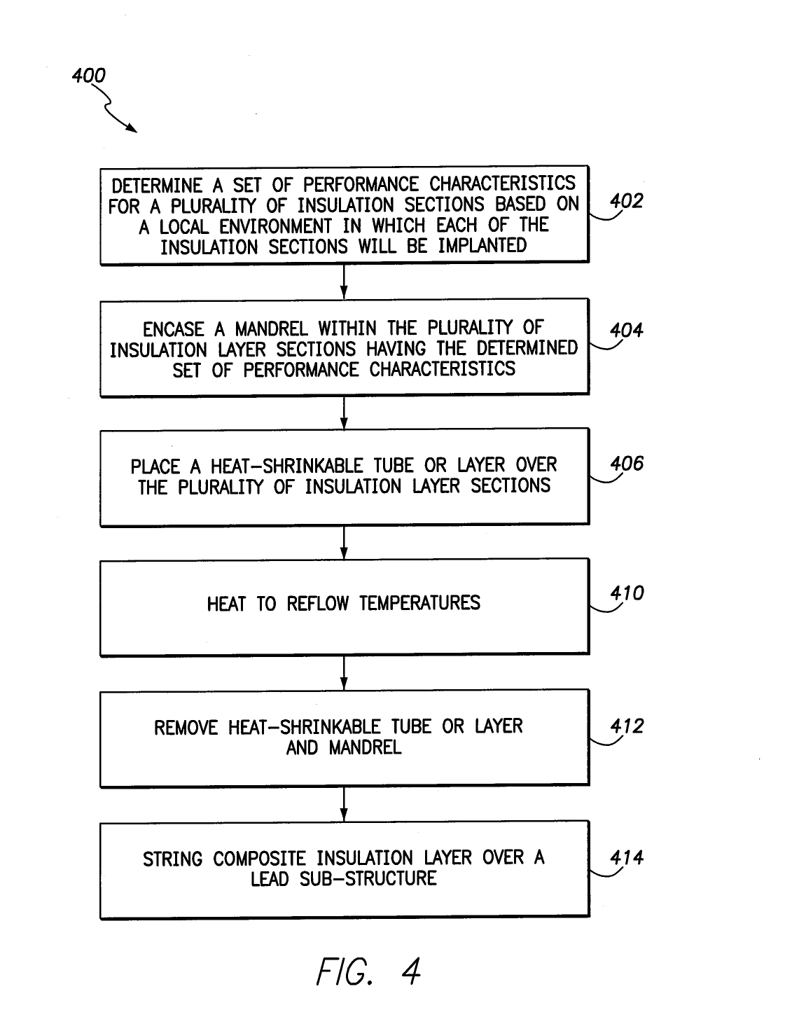

FIG. 3B is the same view as FIG. 3A with the sections 210 and 212 fused together to form a composite insulation layer (the insulation layer 202). The sections 210 and 212 may be fused together, for example, using the operations described with respect to FIG. 4. Once the sections 210 and 212 are fused together, the transition 214 is formed such that the insulation layer 202 transitions seamlessly from the first set of performance characteristics of the first section 210 to the second set of performance characteristics of the second section 212. For example, as shown in the implementation in FIG. 3B, the insulation layer 202 seamlessly transitions from the first section 210, which is thicker-walled and more robust, to the second section 212, which is thinner-walled and more flexible, at the transition 214.

FIG. 3C shows the same view as FIG. 3B with the support mandrel 300 removed. The insulation layer 202 may then be strung over the lead sub-structure, such as the electrical conductors 206, an insulation sub-structure (e.g., a multi-lumen lead body), a helical cable assembly, or other lead components. It will be appreciated by those skilled in the art that although FIGS. 3A-3C show the use of the support mandrel 300 during the manufacturing of the insulation layer 202, the insulation layer 202 may be formed directly on the lead sub-structure. Other manufacturing techniques are also contemplated.

Referring to FIG. 4, example operations 400 for manufacturing the insulation layer 202 using reflow techniques are described. In one implementation, a determining operation 402 identifies one or more local implant environments along a path the implantable lead 200 will traverse and determines the performance characteristics warranted by each of the local implant environments and/or by a placement approach. The determining operation 402 defines a set of performance characteristics for each of a plurality of insulation layer sections based on the local environment in which each insulation layer section will be implanted, as described herein. The determining operation 402 obtains the plurality of insulation layer sections, each having a set of performance characteristics optimized for the local environment in which the insulation layer section will be implanted. Stated differently, the determining operation 402 determines a set of performance characteristics warranted for a particular local implant environment and/or placement approach and obtains an insulation layer section having a set of performance characteristics optimized for the local implant environment and/or placement approach. The determining operation 402 similarly obtains insulation layer sections optimized for other particular local implant environments.

An encasing operation 404 encases a support mandrel or core rod within the plurality of insulation layer sections obtained during the determining operation 402. Alternatively, the encasing operation 404 may encase a lead sub-structure with the plurality of insulation layer sections obtained during the determining operation 402. The encasing operation 404 positions each of the insulation layer sections relative to each other based on a profile of the insulation layer needed to meet the demands of each of the one or more local implant environments. Stated differently, the encasing operation 404 positions the plurality of insulation layer sections such that the insulation layer that is formed will result in each of the insulation layer sections being implanted in the local environment for which that insulation layer section is optimized.

A placing operation 406 places a heat-shrinkable layer or tube over the plurality of insulation layer sections. In some implementations, the heat-shrinkable layer is a polymeric material, such as fluorinated ethylene propylene (FEP). A heating operation 410 heats the heat-shrinkable layer and the components encased by the heat-shrinkable layer to reflow temperatures. Specifically, the heating operation 410 heats the heat-shrinkable layer and the components encased by the heat-shrinkable layer until the plurality of insulation layer sections reach a melt-flow temperature, which causes the plurality of insulation layer sections to fuse together to form a composite insulation layer having one or more seamless transitions along the length of the insulation layer between each of the insulation sections. Once the temperatures cool, a removing operation 412 removes the heat-shrinkable layer and the support mandrel, where applicable. Unless the operations 404-412 were performed directly on the lead sub-structure, a stringing operation 414 strings the composite insulation layer over the lead sub-structure.

In embodiments where the insulation material is a thermoset material that does not melt-flow, the operations as depicted in FIGS. 3A-4 may be modified accordingly. For example, the insulation material may be a thermally-cured silicone elastomer (such as Dow Corning Silastic Q7-4780 medical grade ETR elastomer). A thin-walled extruded, but un-vulcanized silicone tube (i.e., the thermally-cured silicone is in the un-cured/green state) and a thick-walled extruded, but un-vulcanized silicone tube (i.e., the thermally-cured silicone is still in the uncured/green state) are placed over the support mandrel 300 similar to the respective tubes 212, 210 depicted in FIG. 3A. The two un-cured tubes are strung together end-to-end on the support mandrel. A heat shrink tube made of, e.g., FEP, is placed over the strung-together thin and thick insulation layers similar to as described above with respect to step 406 of FIG. 4. The silicone segments are pressed/diffused together at the transition under pressure applied by the heat-shrink tube and then allowed to vulcanize (i.e., cure) by applying adequate heat, thereby forming a composite lead body insulation that has a thin flexible segment joined to a thick robust segment by a smooth transition. Unless the preceding manufacturing operations were performed directly on the lead sub-structure, a stringing operation strings the composite insulation layer over the lead sub-structure.

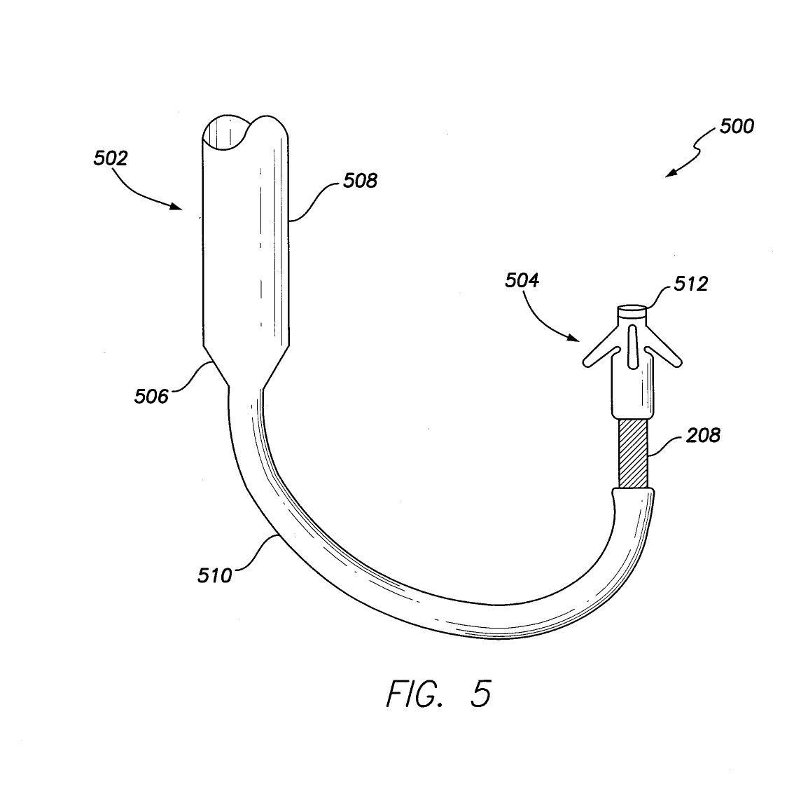

FIGS. 5-10 illustrate specific example implementations of the implantable lead optimized for a specific implant environments and/or placement approaches. Turning to FIG. 5, an implementation of a right atrial lead 500 is shown. Placement of the right atrial lead 500 in the right atrium warrants a proximal end 502 that is relatively robust and a distal end 504 that is relatively flexible. As such, the right atrial lead 500 includes an insulation layer having a transition 506 along the length of the insulation layer between the proximal end 502 and the distal end 504. The transition 506 is seamless between a first section 508 that is thicker and consequently more robust and a second section 510 that is thinner and thus more flexible. As shown in FIG. 5, the transition 506 provides a seamless change in diameter from the thicker, robust first section 508 to the thinner, flexible second section 510. Therefore, the right atrial lead 500 includes a plurality of sections 508 and 510, optimized for local environments in which the sections 508 and 510 will be implanted for stimulation of the right atrium using the electrodes 208 and 512.

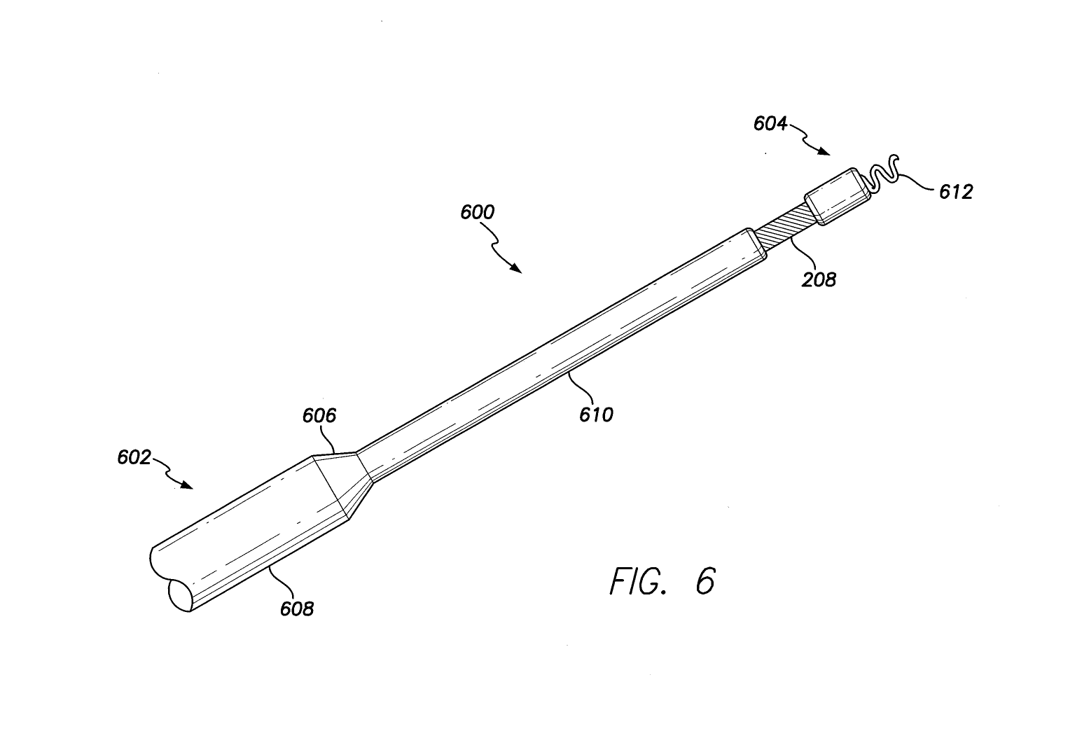

FIG. 6 shows an implementation of a right ventricular lead 600, which includes an insulation layer having performance characteristics corresponding to local implant environments encountered during implant in the right ventricle. Specifically, the right ventricular lead 600 includes a proximal end 602 that is relatively robust and a distal end 604 that is relatively flexible. As such, the right ventricular lead 600 includes an insulation layer having a transition 606 along the length of the insulation layer between the proximal end 602 and the distal end 604. The transition 606 is seamless between a first section 608 that is thicker and consequently more robust and a second section 610 that is thinner and thus more flexible. As shown in FIG. 6, the transition 606 provides a seamless change in diameter from the thicker, robust first section 608 to the thinner, flexible second section 610. Once the distal end 604 is positioned in a target local implant environment, the distal end 604 may be secured using a rotatable fixation helix 612, which may serve as an electrode or an anchoring mechanism for the lead distal end 604.

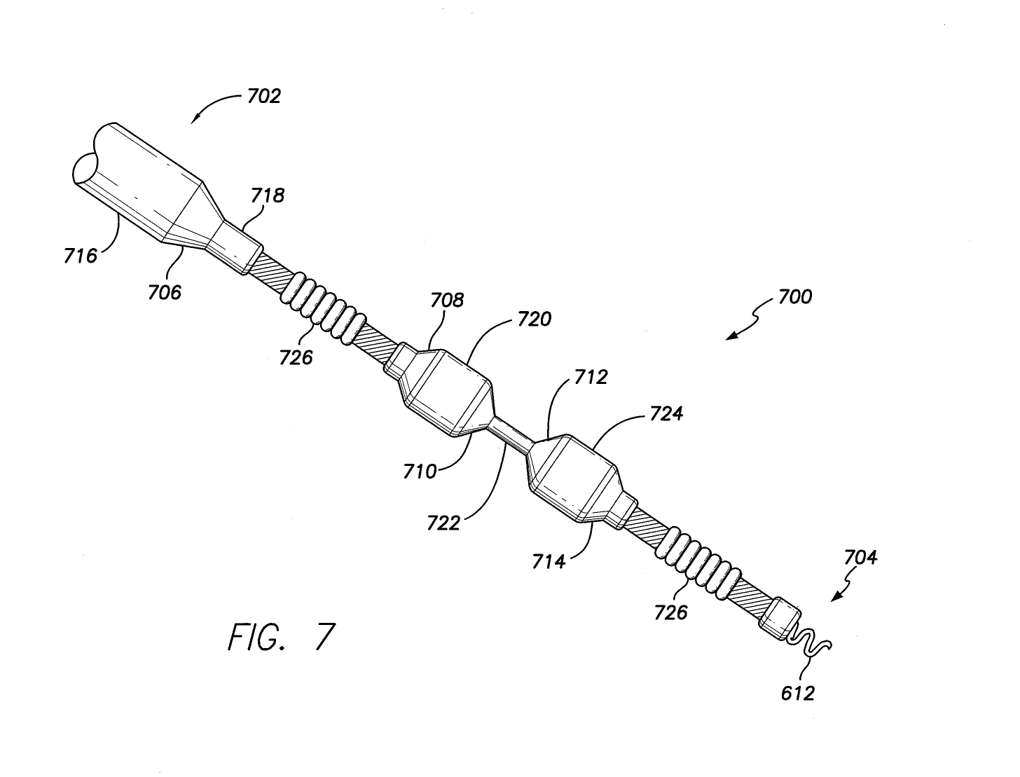

As can be understood from FIG. 7, an implementation of a right ventricular defibrillation lead 700 includes an insulation layer extending from a proximal end 702 to a distal end 704 having a variety of performance characteristics corresponding to local implant environments encountered during implant in the right ventricle. Accordingly, the right ventricular defibrillation lead 700 includes a plurality of transitions 706, 708, 710, 712, and 714 along the length of the insulation layer between the proximal end 702 and the distal end 704. Specifically, the transition 706 is a seamless diameter change from a thicker, robust section 716 to a thinner section 718 positioned relative to a superior vena cava (SVC) shock coil 726. The transitions 708 and 712 provide a seamless change to thicker, abrasion resistant sections 720 and 724, with the transition 710 being a seamless transition from the thicker abrasion resistant section 720 to a thin, flexible section 722, which functions as a buckle point to increase flexibility between the SVC shock coil 726 and the right ventricle shock coil 728. Finally, the transition 714 transitions to the thinner distal end 704.

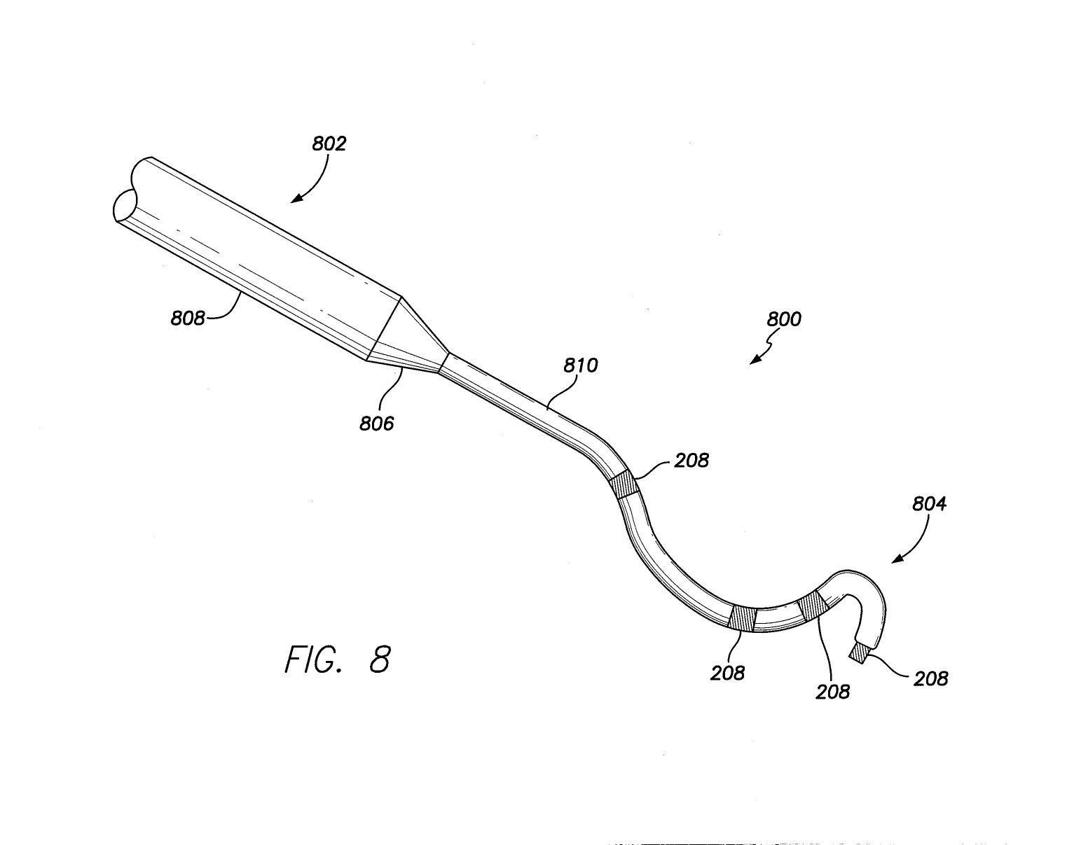

Turning to FIG. 8, an implementation of a left ventricular transvenous cardiac resynchronization therapy (“CRT”) lead 800 is shown. Placement of the left ventricular transvenous CRT lead 800 warrants a proximal end 802 that is robust and easy to maneuver (e.g., push, torque, and otherwise handle) and a distal end 804 that is flexible and trackable. As such, the left ventricular transvenous CRT lead 800 includes an insulation layer having a transition 806 along the length of the insulation layer between the proximal end 802 and the distal end 804. The transition 806 is seamless between a first section 808 that is thicker and has the performance characteristics warranted for the proximal end 802 and a second section 810 that is thinner and has the performance characteristics warranted for the distal end 804. As shown in FIG. 8, the transition 806 provides a seamless change in diameter from the thicker first section 808 to the thinner second section 810. In a specific implementation, the transition 806 provides a seamless change from the first section 808 having a diameter of approximately 0.060 inches to the second section 810 having a diameter of approximately 0.056 inches. In some implementations, the ventricular transvenous CRT lead 800 includes a helical cable sub-structure onto which the insulation layer is formed using the operations 400, as described with respect to FIG. 4.

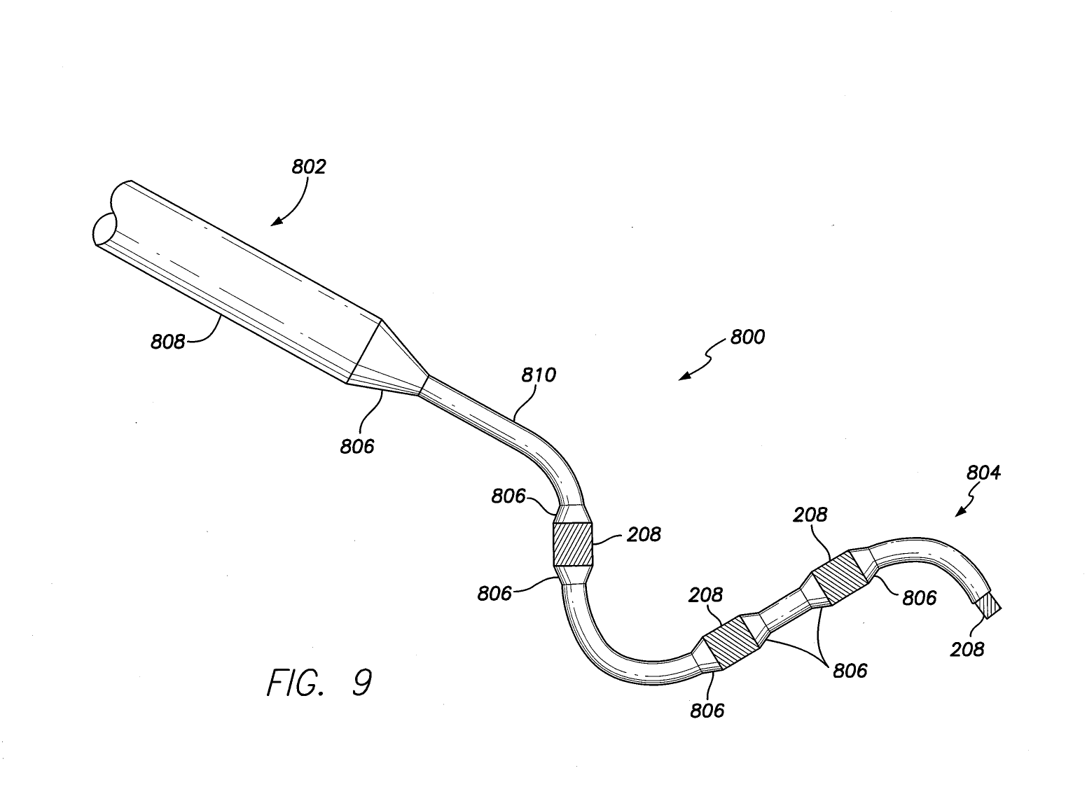

As can be understood from FIG. 9, which depicts a lead similar to that of FIG. 8, the lead body can transition to enlarged diameters at the ring electrodes distal the tip electrode. In other words, as can be understood from FIG. 9, the lead body outside diameter is increased at each of the ring electrodes 208 as compared to the lead body outside diameter just distal or proximal of each ring electrode. A seamless, smooth transition 806 as already described herein is present just distal and proximal of each ring electrode. These local increases in lead-body outside diameter at each of the three ring electrodes distal the tip electrode promotes electrode-tissue contact since the ring electrode can stand proud of the immediately adjacent lead body. Also, the smooth transitions distal and proximal each ring electrode provides strain relief to conductor terminations at the respective ring electrode. While transitions to/from local increases in the outside diameter of the lead body at ring electrodes are illustrated in FIG. 9, in should be noted that such local increased lead body diameters and the accompanying transitions to/from may be provided for any pertinent feature or component on the lead that may benefit from this seamless method of achieving desirable positive contact with the target tissue. Similarly the opposite is also achievable in a case where it is beneficial to avoid contact of a lead component or feature with surrounding tissue. In other words, were appropriate for the component being supported on the lead body where contact between the component and the surrounding tissue should be limited, there may be local decreased lead body outside diameters with corresponding seamless transitions to/from the respective local decreased lead body outside diameter.

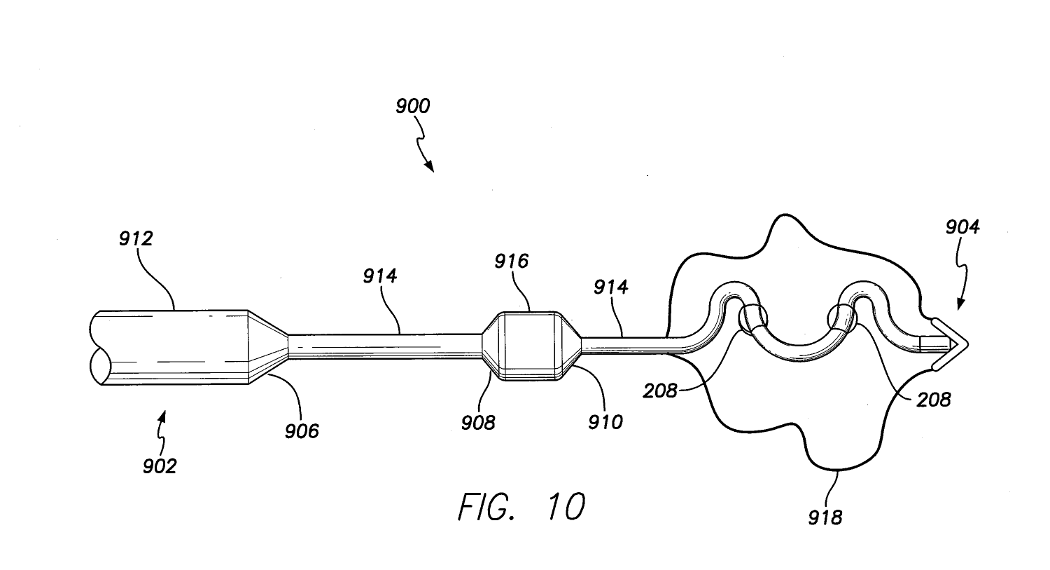

FIG. 10 illustrates an implementation of an epicardial lead 900, which includes an insulation layer extending from a proximal end 902 to a distal end 904 having a variety of performance characteristics corresponding to local implant environments encountered during implant in the intrapericardial space of the patient heart 102. Specifically, the distal end 904 warrants increased trackability through an introducer and a reduced risk of dislodgment, and the proximal end 902 needs increased abrasion and crush resistance based on the tunneling path and harsh local implant environments as the epicardial lead 900 wraps around the ribs, for example, while moving out of the thoracic cavity. Accordingly, the epicardial lead 900 includes a plurality of transitions 906, 908, and 910 along the length of the insulation layer between the proximal end 902 and the distal end 904. Specifically, the transition 906 is a seamless diameter change from a proximal thicker, robust section 912 to a thinner section 914, which provides increased flexibility and stability. The transitions 908 and 910 provide seamless changes to and from a distal thicker, robust section 916 having a local diameter increase to ensure positive contact of fixation features located on section 916 with the epicardium and to increase stability at the final implant location. In a specific implementation, the proximal thicker, robust section 912 is approximately 0.072 inches in diameter, the thinner, flexible section 914 is approximately 0.062 inches in diameter, and the distal thicker, robust section 916 is approximately 0.072 inches in diameter. In another specific implementation, the thinner, flexible section 914 is approximately 20 inches in length with the distal, thicker robust section 916 being approximately 1 inch in length. In some implementations, the insulation layer is formed using the operations 400 directly on the lead sub-structure of the epicardial lead 900, as described with respect to FIG. 4.

Various modifications and additions can be made to the exemplary embodiments discussed without departing from the spirit and scope of the presently disclosed technology. For example, while the embodiments described above refer to particular features, the scope of this disclosure also includes embodiments having different combinations of features and embodiments that do not include all of the described features. Accordingly, the scope of the presently disclosed technology is intended to embrace all such alternatives, modifications, and variations together with all equivalents thereof.

1. An implantable lead optimized for an implant environment, the implantable lead comprising:
an insulation layer having one or more transitions along a length of the insulation layer from a proximal end to a distal end, each of the transitions being a seamless change from a section of the insulation layer having a set of performance characteristics to another section of the insulation layer having a different set of performance characteristics;
wherein a first section of the insulation layer is disposed at a distal portion of the insulation layer and a second section of the insulation layer is disposed proximal to the first section of the insulation layer, wherein the first section of the insulation layer has a first set of performance characteristics including a first wall thickness and the second section of the insulation layer has a second set of performance characteristics including a second wall thickness, and wherein the second wall thickness is greater than the first wall thickness.

2. The implantable lead of claim 1, wherein the performance characteristics further include material type and durometer.

3. (canceled)

4. (canceled)

5. (canceled)

6. (canceled)

7. (canceled)

8. (canceled)

9. (canceled)

10. The implantable lead of claim 1, wherein the one or more transitions includes a first transition from a first section to a second section having a local diameter decrease.

11. The implantable lead of claim 1, further comprising a component supported on the lead body and the component is located at the second section.

12. The implantable lead of claim 11, wherein the component includes a ring electrode, sensor or fixation mechanism.

13. (canceled)

14. An implantable lead insulation layer comprising:
a first section having a first set of performance characteristics;
a second section having a second set of performance characteristics that is different from the first set of performance characteristics; and
a first transition between the first section and the second section, the first transition preventing the first set of performance characteristics from compromising the second set of set of performance characteristics.

15. The implantable lead insulation layer of claim 14, wherein the transition is seamless.

16. The implantable lead insulation layer of claim 14, wherein the first and second sets of performance characteristics include wall thickness, material type, and durometer.

17. The implantable lead insulation layer of claim 14, wherein the first set of performance characteristics includes a first wall thickness and the second set of performance characteristics includes a second wall thickness, the first wall thickness being different than the second wall thickness.

18. The implantable lead insulation layer of claim 17, wherein the first wall thickness is greater than the second wall thickness.

19. The implantable lead insulation layer of claim 18, wherein the first wall thickness is proximal the second wall thickness.

20. The implantable lead insulation layer of claim 14, wherein the first set of performance characteristics includes a first material type and the second set of performance characteristics includes a second material type, the first material type being different than the second material type.

21. The implantable lead insulation layer of claim 20, wherein the first material type is robust relative to the second material type and the second material type is flexible relative to the first material type.

22. The implantable lead insulation layer of claim 21, wherein the first material type is proximal the second material type.

23. The implantable lead insulation layer of claim 14 further comprising:
a second transition to a third section having a third set of performance characteristics.

24. The implantable lead insulation layer of claim 23, wherein the third set of performance characteristics includes a local diameter increase.

25. The implantable lead insulation layer of claim 23, wherein the third set of performance characteristics includes abrasion resistance.

26. A method for manufacturing an implantable lead optimized for an implant environment, the method comprising:
obtaining a plurality of insulation layer sections, each insulation layer section having a set of performance characteristics based on a local environment in which the insulation layer section is to be implanted;
positioning the plurality of insulation layer sections relative to each other; and
fusing the plurality of insulation layer sections together such that one or more transitions are formed along a length of a composite insulation layer.

27. The method claim 26, wherein the plurality of insulation layer sections are fused together using reflow techniques.

28. The method of 26, wherein the plurality of insulation layer sections each include a thermoset material that is not capable of melt-reflow.

29. The method of claim 26, wherein the performance characteristics include wall thickness, material type, and durometer.

30. The method of claim 26, wherein the one or more transitions includes a first transition from a first insulation layer section having a first set of performance characteristics to a second insulation layer section having a second set of performance characteristics that are different than the first set of performance characteristics.

31. The method of claim 30, wherein the first set of performance characteristics includes a first wall thickness and the second set of performance characteristics includes a second wall thickness, the first wall thickness being greater than the second wall thickness.

32. The method of claim 31, wherein the first wall thickness is proximal the second wall thickness.

33. The method of claim 30, wherein the first set of performance characteristics includes a first material type and the second set of performance characteristics includes a second material type, the first material type being robust relative to the second material type and the second material type being flexible relative to the first material type.

34. The method of claim 33, wherein the first material type is proximal the second material type.

35. The method of claim 26, wherein at least one transition of the one or more transitions is seamless.
